(19) United States
(12) Patent Application Publication
(10) Pub. No.: US 2009/0118558 A1
(43) Pub. Date: May 7, 2009

(54) Dehydration Process

(75) Inventors: Martin Philip Atkins, Middlesex (GB); Martyn John Earle, Lisburn (GB); Thomas Stephen Wittrig, Wheaton, IL (US)

Correspondence Address: NIXON & VANDERHYE, PC, 901 NORTH GLEBE ROAD, 11TH FLOOR, ARLINGTON, VA, 22203, US

(73) Assignee: BP p.l.c., London (GB)

(21) Appl. No.: 11/988,871

(22) PCT Filed: Jul. 20, 2006
(86) PCT No.: PCT/GB2006/002732

§ 371 Date: Jan. 16, 2008

Related U.S. Application Data
(60) Provisional application No. 60/702,614, filed on Jul. 27, 2005.

(51) Int. Cl. C07C 1/20

(52) U.S. Cl. 585/639

(57) ABSTRACT

A process for producing an olefin and/or an ether is described, which comprises heating an alcohol in the presence of an acidic ionic compound which exists in a liquid state at a temperature of below 150° C.

This invention relates to a process for dehydrating alcohols to give olefins and/or ethers.

The dehydration of alcohols to produce olefins and/or ethers is well known in the art. For example, ethanol, propanol or isopropanol can be dehydrated to form ethylene or propylene. At least some ether is generally produced as a by-product. In the case of methanol, the product is predominantly dimethyl ether. The generation of olefins and ethers by such dehydration reactions is becoming commercially more important for a variety of reasons; for example, alcohols are frequently easier and safer to transport than the corresponding olefins and ethers.

The dehydration of alcohols can be carried out commercially using catalysts such as zeolites at elevated temperatures. The temperature employed is frequently around 300 to 350° C. As well as zeolites, catalysts used to dehydrate alcohols include alumina (aluminium oxide), aluminophosphates and silicoaluminophosphates, activated carbon, and crystalline ytterbium aluminium borate.

It is an object of the present invention to provide a process for preparing olefins and/or ethers by the dehydration of alcohols.

Accordingly the present invention provides a process for producing an olefin and/or an ether which comprises heating an alcohol in the presence of an acidic ionic compound which exists in a liquid state at a temperature of below 150° C.

The ionic compound which exists in a liquid state at a temperature of below 150° C. will hereinafter be referred to as an ionic liquid. Preferably, the ionic liquid will be a compound that exists in a liquid state at a temperature of below 100° C. In the liquid phase, the degree of ionisation of the ionic liquid will generally be at least 90%, preferably at least 95%, more preferably at least 98%, and most preferably at least 99%.

Preferably an ionic liquid which is stable (i.e. is not significantly irreversibly decomposed) in the presence of water is used, as water is produced as a by-product of the reaction.

Alcohols suitably employed as reactants in the present invention may be primary, secondary or tertiary alcohols, for example those containing 1 to 50 carbon atoms, preferably 1 to 20, more preferably 1 to 8 carbons atoms, for example methanol, ethanol, a propanol, a butanol or a pentanol. The dehydration of alkanols, and especially ethanol, is particularly valuable commercially. Aromatic alcohols having the formula Ar—[(CH)₂]ₙ—OH wherein n=1 to 40, preferably 1 to 20 and Ar represents an aryl group, may be used. A mixture of alcohol reactants may be employed.

For methanol, any tertiary alcohol, or an aromatic alcohol of the above formula where n=1, the product will be predominantly an ether. For most other alcohols, the product may be either an ether or an olefin or a mixture, the exact composition depending upon the reaction conditions and the particular reagents employed. For higher alcohols, mixtures of olefins and/or mixtures of ethers are likely to be produced. In general, where either ether or olefin products may be obtained in principle, using a higher temperature tends to lead to increased production of olefins and decreased production of ethers.

The process of the invention is carried out by heating at a temperature sufficiently high to cause at least some dehydration of the alcohol to olefin and/or ether, and suitably at a temperature at which dehydration proceeds at a commercially acceptable rate. Suitable temperatures generally lie in the range 100 to 400° C., preferably 100 to 250° C., with temperatures of higher than 200° C. generally being preferred when the desired product is an olefin. Thus, the ionic liquid used should be substantially stable at the reaction temperature. Excessively high temperatures should be avoided as this can lead to undesired oligomerization and/or polymerisation of the product.

Heating may be carried out by any suitable method, for example by direct heating or by irradiating the reaction mixture with microwave radiation.

The pressure is preferably maintained in the range from 0.1 to 100 bar absolute, preferably 0.5 to 10 bar absolute, most preferably from 1 to 4 bar absolute. Generally, it is preferred that the pressure is such that the olefin and/or ether product, and the co-produced water, are in a gaseous state such that a gaseous (vapour) phase comprising the olefin and/or ether product and the co-produced water separates from a liquid phase comprising the ionic liquid. The reaction can be carried out with the alcohol reactant in either the liquid or gaseous phase. The co-produced water and any vaporised alcohol reactant may then be condensed out from the olefin and/or ether product. However, where the olefin and/or ether product is liquid or easily condensed to a liquid, the product, co-produced water and any vaporised alcohol reactant can, if desired, be separated by any suitable method, for example fractional distillation or azeotropic distillation. If desired, the produced olefin and/or ether can be dried and/or subjected to purification. For example, the olefin and/or ether can be conducted through one or more beds of molecular sieve to remove traces of co-produced water and/or other impurities.

The ionic liquid acts as a catalyst for the reaction, and may be presented in homogeneous or heterogeneous form. When using a homogeneous ionic liquid catalyst, the ionic liquid can be employed as a distinct liquid phase (for example, as a pool of liquid), as a spray (i.e. discrete droplets of liquid), or as a flowing liquid. Preferably, the olefin and/or ether product and the co-produced water are separated from the homogeneous ionic liquid catalyst as a gaseous (vapour) phase. Where the ionic liquid is employed as a spray, it is preferred that the droplets of ionic liquid are allowed to coalesce so that the gaseous phase can be readily separated from the liquid phase.

Alternatively, a heterogeneous catalyst may comprise an ionic liquid supported on a suitable support material. Suitably, the support material is substantially insoluble in the ionic liquid. Examples of preferred support materials include silica, alumina, silica-alumina, pumice, kieselguhr, glass beads, and diatomaceous earth materials. Where a reaction employing a heterogeneous catalyst is to be carried out by feeding liquid alcohol and/or removing liquid products (liquid olefin and/or liquid ether and liquid co-produced water) from the reaction zone, the ionic liquid is preferably selected from those which are substantially insoluble in the liquid alcohol and the liquid products. This is to prevent the ionic liquid from being washed off the support material. However, when the alcohol reactant, the olefin and/or ether product and co-produced water are maintained in a gaseous phase when contacted with the supported ionic liquid, it is not necessary to select an ionic liquid that is insoluble in the alcohol reactant, the olefin and/or ether product and the co-produced water.

In general, the use of a homogeneous catalyst is preferred.

The process of the invention may if desired be carried out in the presence of a solvent. Suitable solvents are those which are substantially inert in the presence of catalyst, for example alkanes, haloalkanes, and inert ethers (for example the product ether) or ketones may be used.

The ionic liquid may be used alone as the dehydration catalyst, or it may be used together with a compound capable of imparting further acidity to the reaction mixture, i.e. a Bronsted acid or Lewis acid. Anhydrous mineral acids are preferred, especially an acid selected from phosphoric, sulfuric, and selenic acid. Examples of Lewis acids include aluminium chloride, iron (III) chloride, boron trifluoride, niobium pentachloride and ytterbium (III) triflate.

The reaction may be carried out continuously, semi-continuously or discontinuously. For example, the reaction can be carried out in a continuous stirred tank reactor. The alcohol reactant can be introduced intermittently or continuously, or as a single batch, into the stirred ionic liquid.

The present invention has a variety of potential advantages in comparison with the prior art processes. Generally the present invention operates at lower temperatures than prior art processes resulting in energy saving, production of fewer by-products and/or production of lower quantities of such by-products. This also allows cheaper materials to be used for the fabrication of plant equipment (for example, a stainless steel reactor or a glass-lined reactor).

The ionic liquid may be represented by the formula [C]⁺[An]⁻ where [C]⁺ is a cation that forms a liquid salt with anion [An]⁻, and must have acidic properties. It may contain an acidic anion and/or an acidic cation, i.e. it may comprise an acidic cation and a neutral anion, or a neutral cation and an acidic anion, or both an acidic cation and an acidic anion, or mixtures thereof. Mixtures of two or more different ionic liquids may be used.

An acidic cation preferably has the formula Cat⁺-Z-Acid wherein Cat⁺ is a cationic species; Z is a linking group joining Cat⁺ and Acid which may be a covalent bond or a group (especially an alkyl group) containing 1 to 30, especially 1 to 10, for example 2 to 8, and especially 3 or 4, carbon atoms and optionally one, two or three oxygen atoms; and Acid is an acidic moiety.

Acid is preferably selected from —SO₃H, —CO₂H, HSO₃-Ph-, HSO₃-Ph(R)—, —PO(OH)₂, —PO(OH), and —PO. R.(OH); wherein R is, for example, a C₁ to C₆ alkyl or haloalkyl group or an aryl group bearing one or more inert substituents.

An acidic cation may for example be a quaternary ammonium or phosphonium cation of the general formula:

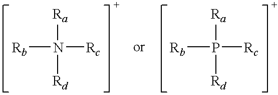

where each of Rₐ R_{b} R_{c} and R_{d} are independently selected from H, an alkyl group having from 1 to 30, preferably from 1 to 10, for example 2 to 8, especially 3 or 4, carbon atoms, which may be optionally interrupted by 1, 2 or 3 oxygen atoms, an aryl group, or a group —Z-Acid as defined above, at least one of Rₐ R_{b} R_{c} and R_{d} representing a group —Z-Acid.

Cat⁺ may for example comprise or consist of a heterocyclic ring structure selected from imidazolium, pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium, diazabicyclo[5,4,0]undecenium, diazabicyclo[4,3,0]nonenium and pyrrolidinium.

Preferably, Cat⁺ comprises or consists of a heterocyclic ring structure selected from pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium, diazabicyclo[5,4,0]undecenium, diazabicyclo[4,3,0]nonenium and pyrrolidinium.

More preferably, Cat⁺ comprises or consists of a heterocyclic ring structure selected from imidazolium, pyridinium, pyrazolium, isothiazolinium, triazolium, tetrazolium, piperidinium, morpholinium, diazabicyclo[5,4,0]undecenium, diazabicyclo[4,3,0]nonenium, and pyrrolidinium.

Preferably, Cat⁺-Z-Acid is selected from:

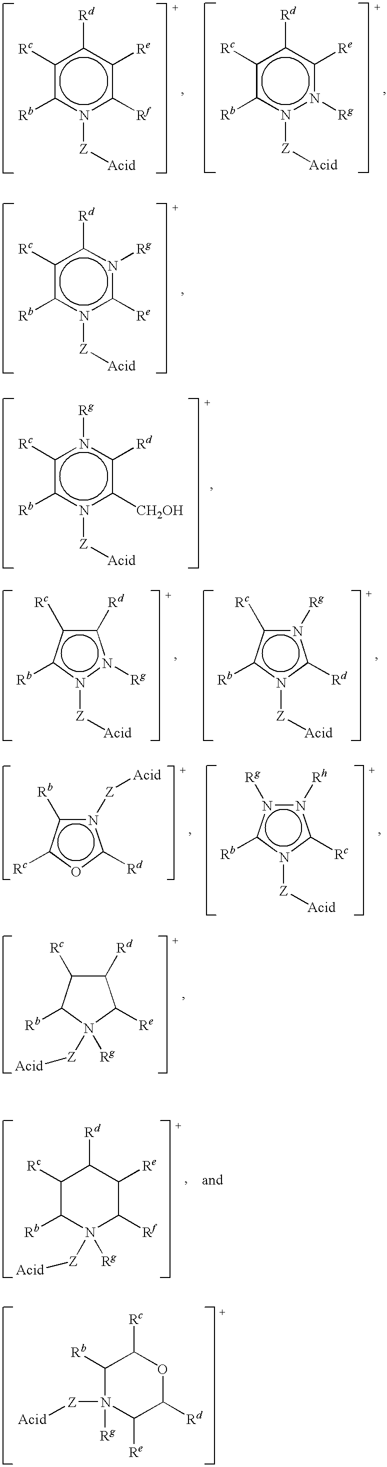

wherein Acid and Z are as defined above; and R^{b}, R^{c}, R^{d}, R^{e}, R^{f}, R^{g} and R^{h} are each independently selected from hydrogen, a C₁ to C₄₀ alkyl group, a C₃ to C₈ cycloalkyl group, or a C₆ to C₁₀ aryl group, wherein said alkyl, cycloalkyl or aryl groups are unsubstituted or may be substituted by one to three groups selected from C₁ to C₆ alkoxy, C₆ to C₁₀ aryl, CN, OH, NO₂, C₇ to C₃₀ aralkyl, and C₇ to C₃₀ alkaryl, or any two of R^{b}, R^{c}, R^{d}, R^{e} and R^{f} attached to adjacent carbon atoms may form a methylene chain —(CH₂)_{q}— wherein q is from 3 to 6.

In one preferred embodiment, Cat⁺-Z-Acid is:

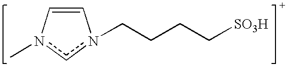

Further, an acidic anion may for example be selected from [HSO₄]⁻, [H₂PO₄]⁻, [HPO₄]²⁻, and [HX₂]⁻ wherein X=F, Cl, Br or I; especially [HCl₂]⁻, [HF₂]⁻, [HSO₄]⁻, and [H₂PO₄]⁻.

Binary acidic ionic liquids containing anions of the type [Hₙ(Y)ₙ₊₁]⁻, for example [H(CF₃SO₂)₂]⁻, made by mixing an acidic compound, which may be either a Bronsted acid or a Lewis acid with a suitable anion, may also be used. This may be represented by the equations:

For Bronsted acids:

HX+[Cation][Anion]=[Cation][Anion-H-X]

m HX+[Cation][Anion]=[Cation][Anion-(H-X)ₘ]

For Lewis acids:

MXₙ+[Cation][Anion]=[Cation][Anion-MXₙ]

m MXₙ+[Cation][Anion]=[Cation][Anion-(MXₙ)ₘ]

where M is a metal and m is the number of mols of acid used. Both these types of ionic liquids are suitable to catalyse the dehydration reactions and can be used with acidic or neutral types of cations. Any acid HX may be used for this process, but strong mineral acids or strong organic acids are preferred, for example sulfonic acids, fluorinated sulfonic acids, phosphoric acids, hydrogen sulfonamides (H—N(SO₂)₂R), especially HN(SO₂CF₃)₂ and HN(SO₂C₂F₅)₂, alkylsulfonic acids and haloacids.

A Lewis acid (MXₙ) can be any metal halide or metal complex that exhibits Lewis acidity. Preferred are metals such as transition metal compounds, Group 13, 14, 15, 16 metals or semi metals, and lanthanide or actinide metals. Of these, Group 13 metals or other trivalent metals are preferred and most preferred are aluminium, gallium and indium compounds. X is preferable a halide or oxygenated ligand, or a nitrogen ligand. Most preferable X is a halide, for example chloride.

The anions used to form such a binary compound are preferably those that give rise to a strong conjugate acid. These can be selected from the following non exclusive list: [C(CN)₃]⁻, [NTf₂]⁻, [OTf]⁻, [R—SO₃]⁻, [R₂PO₂]⁻, [Cl]⁻, [Br]⁻, and [I]⁻, wherein R is C₁ to C₆ alkyl, C₆ to C₁₀ aryl, or C₇ to C₁₂ alkaryl, for example [Me—SO₃]⁻, [Ph-SO₃]⁻ and [Me-Ph-SO₃]⁻.

Where the ionic liquid comprises an acidic anion, any neutral cation may be used, provided that the resulting ionic compound has a suitable melting point. One class of neutral cations correspond to the acidic quaternary ammonium or phosphonium cations defined above, save that no acid group is present, i.e. cations of the general formula NRₐR_{b}R_{c}R_{d}⁺ or PRₐR_{b}R_{c}R_{d}⁺ in which each of Rₐ R_{b} R_{c} and R_{d} is independently selected from H, an alkyl group having from 1 to 30, preferably from 1 to 10, for example 2 to 8, especially 3 or 4, carbon atoms, which may be optionally interrupted by 1, 2 or 3 oxygen atoms, or an aryl group.

A further group of neutral cations comprise or consist of a heterocyclic ring structure selected from imidazolium, pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium, diazabicyclo[5,4,0]undecenium, diazabicyclo[4,3,0]nonenium, and pyrrolidinium.

Preferably, a neutral cation preferably comprises or consists of a heterocyclic ring structure selected from pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium, diazabicyclo[5,4,0]undecenium, diazabicyclo[4,3,0]nonenium, and pyrrolidinium.

More preferably, a neutral cation comprises or consists of a heterocyclic ring structure selected from pyridinium, pyrazolium, thiazolium, pyrimidinium, piperazinium, piperidinium, morpholinium, quinolinium, isoquinolinium, diazabicyclo[5,4,0]undecenium, diazabicyclo[4,3,0]nonenium, and pyrrolidinium.

Preferably a neutral cation is selected from:

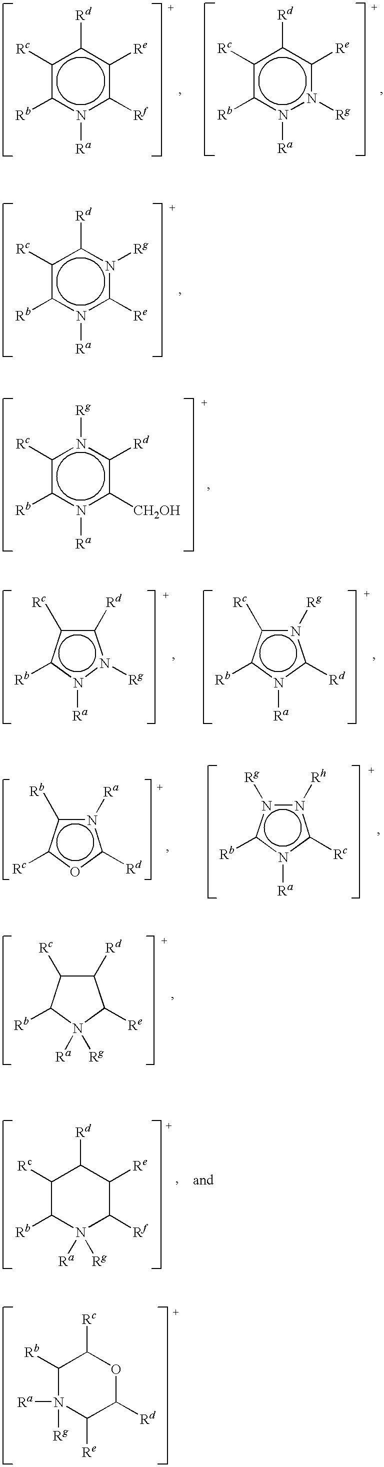

wherein R^{a}, R^{b}, R^{c}, R^{d}, R^{e}, R^{f}, R^{g} and R^{h} are each independently selected from hydrogen, a C₁ to C₄₀ alkyl group, a C₃ to C₈ cycloalkyl group, or a C₆ to C₁₀ aryl group, wherein said alkyl, cycloalkyl or aryl groups are unsubstituted or may be substituted by one to three groups selected from C₁to C₆ alkoxy, C₆ to C₁₀ aryl, CN, OH, NO₂, C₇ to C₃₀ aralkyl and C₇ to C₃₀ alkaryl, or any two of R^{b}, R^{c}, R^{d}, R^{e} and R^{f} attached to adjacent carbon atoms form a methylene chain —(CH₂)_{q}— wherein q is from 3 to 6.

Where the ionic liquid comprises an acidic cation, a neutral anion may for example be a carboxylate such as trifluoroacetate, hydrogen sulfate, sulfonate, phosphinate, triflamide (amide), triflate, dicyanamide, oxide (phenoxide) or halide anionic species. Preferably, the neutral anion is selected from [C(CN)₃]⁻, [NTf₂]⁻, [OTf]⁻, [R—SO₃]⁻, [R₂PO₂]⁻, [Cl]⁻, [Br]⁻ and [I]⁻, wherein R is C₁ to C₆ alkyl, C₆ to C₁₀ aryl, or C₇ to C₁₂ alkaryl, for example [Me—SO₃]⁻, [Ph-SO₃]⁻ and [Me-Ph-SO₃]⁻.

Specific examples of suitable cations, [C]⁺, include choline ([HOCH₂CH₂N(CH₃)₃]⁺), 1-alkyl-3-methylimidazolium cations (where alkyl is a C₆ to C₁₈ alkyl group, preferably, hexyl, octyl, decyl, dodecyl, hexadecyl, or octadecyl), and 4-(3-methylimidazolium)-butanesulfonate (MBIS). Examples of suitable anions, [An]⁻, include dihydrogenphosphate, hydrogensulfate, trifluromethanesulfonate (CF₃SO₃⁻), bistrifluoromethanesulfonylamide ([(CF₃SO₂)₂N]⁻), tosylate (CH₃C₆H₄SO₃⁻) and metal anions such as [MClₘ]⁻ where M is gallium or indium.

Preferred ionic liquids include choline salts, for example choline dihydrogenphosphate or choline hydrogensulfate, hexylmethylimidazolium hydrogensulfate ([C₆mim][HSO₄]), [MIBS][(CF₃SO₂)₂N]), [MIBS][CF₃SO₃] (having a melting point of approximately 50° C.), [MIBS][CH₃C₆H₄SO₃], [MIBS][H₂PO₄] (having a melting point of 84° C.), N-butylpyridinium triflate ([BuPy][OTf]), or 3-(3-methylimidazolium-1-yl)propane-1-sulfonate. It is also possible to use compounds of formula [C]⁺[MClₘ]⁻ wherein M is as defined above and [C]⁺ is any cation that forms a liquid salt with [MClₘ]⁻. [MIBS][(CF₃SO₂)₂N] is preferred.

The present invention is further illustrated with reference to the following Examples.

### EXAMPLE 1

### Butanol Dehydration

Butanol was passed through a 250 mm×3.5 mm column packed with an ionic liquid (25 wt. %) supported on flash silica (2.50 g) at a rate of 5 ml per hour. Reaction temperatures=225, 275, 300, 325, 350 and 375° C. The ionic liquid employed was [choline][H₂PO₄] (hereinafter referred to as “choline dihydrogenphosphate”). Phosphoric acid (H₃PO₄) was used to increase the catalyst activity. The catalyst was prepared by adding a solution of 5 g of ionic liquid in methanol to 15 g flash silica, then adding H₃PO₄ (1.0 g). The choline dihydrogen phosphate was in turn made by reacting choline hydroxide (1 equivalent) with phosphoric acid (3 equivalents). Choline dihydrogen phosphate is insoluble in butanol, thus preventing its loss during the reaction.

The outlet of the column was run through two traps, one at a temperature of 20° C. to collect butanol and water and a second cooled to a temperature of −78° C. to collect butene isomers. The mass of products in the two traps was recorded after 30 minutes collecting the products.

No acid was detected (using pH paper) in the products. Also, no phosphate was present in the products, as measured by ³¹P NMR analysis

Variation of yield and isomer ratio with time for the dehydration of butanol using choline dihydrogen phosphate (2.5 g) supported on flash SiO₂ was determined by NMR analysis. The yields were determined by the water content of the unreacted butanol layer and by weighing the product fractions. The results are recorded in Table 1.

TABLE 1

| | | 1-butene to 2- | |
|---|---|---|---|
| Temperature | % Yield | butene ratio | comments |
| 225 | 38 | Not measured | #1 |
| 275 | 49 | 0.24:0.76 | #2 |
| 300 | 27 | 0.43:0.57 | #3 |
| | 43 | | #1 |
| 325 | 39 | 0.51:0.49 | #3 |
| | 70 | | #1 |
| 350 | 70 | 0.53:0.47 | #3 |
| 375 | 97 | 0.44:0.56 | #3 |
| | 97 | | #1 |

Notes
#1 = NMR yield based on water in butanol analysis.
#2 = Determined by NMR analysis of total product mixture trapped at −78° C.
#3 - Isolated yield (by mass).

### EXAMPLE 2

### Butanol Dehydration

A test was carried out as above using the ionic liquid [C₆mim] [HSO₄] instead of choline dihydrogen phosphate in the absence of H₃PO₄. Although the [C₆mim] [HSO₄] was soluble in butanol and was washed off the column during the reaction, some products were observed.

### EXAMPLE 3

### Butanol Dehydration

A test was carried out using [choline] [hydrogensulfate] (6 g) supported on silica (12 g) with 1 g of added H₂SO₄. This catalyst produced the desired products at lower temperatures than with the choline dihydrogen phosphate system, although the system suffered from serious catalyst leaching and the water/butanol stream was acidic.

### EXAMPLE 4

### Ethanol Dehydration

Choline dihydrogen phosphate was used in the dehydration of ethanol at temperatures of up to 375° C. Approximately 10% of the ethanol was converted to diethyl ether and an unquantified amount of ethylene.

### EXAMPLES 5-6

The ionic liquid [MIBS][NTf₂] was synthesised in accordance with the following reaction scheme where [MIBS]=4-(3-methylimidazolium-1-yl)-butane-1-sulfonic acid and Tf=CF₃SO₂.

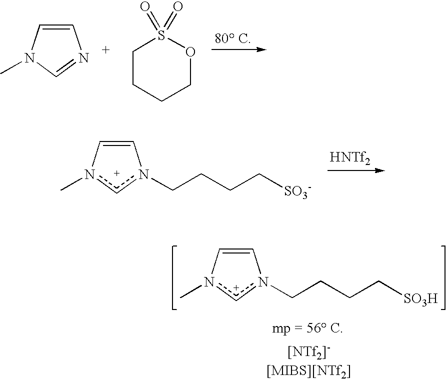

The homogeneously catalysed dehydration of butanol with [MIBS][NTf₂] was carried out in two different ways as shown in the following Examples 5 and 6.

### EXAMPLE 5

A solution of [MIBS][NTf₂] (1% or 2%) in butanol was passed through a heated tube packed with glass beads at up to 350° C. About 45% conversion to dibutylether and less than 5% butenes was obtained. It is envisaged that the yield may be improved by redesigning the apparatus to give longer retention times.

### EXAMPLE 6

In this Example a 2% solution of [MIBS][NTf₂] was heated at various temperatures up to 275° C. in a microwave tube housed in a microwave oven. Heating by microwave radiation provided good controllability of the reaction.

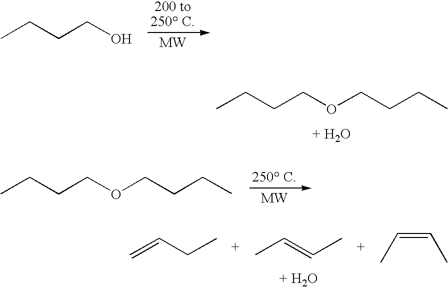

Heating butanol with 2 wt % [MIBS][NTf₂] at 200° C. for 0.5 hours gave a two phase mixture of water (lower layer) and butanol/dibutyl ether (upper layer; 11% yield), with no butene formed. Heating butanol with 2 wt % [MIBS][NTf₂] at 210° C. for 0.5 hours gave a two phase mixture of water (lower layer) and butanol/dibutyl ether (upper layer; 39% yield), with no butene formed. However, heating at 250° C. for 0.5 hours, gave 57% dibutylether and 9% butenes (15.3% but-1-ene and 84.7% but-2-ene as 1:1 mixture of cis- and trans-isomers). Heating at 250° C. for 4 hours resulted in a butanol to dibutylether ratio of 64:27. The ionic liquid had a tendency to dissolve in the layer of co-produced water. However, the ionic liquid was recovered by evaporating off the water.

### EXAMPLES 7 to 11

These examples were carried out by dropping an alcohol via a tap from a dropping funnel onto 10 to 20 mmol of hot ionic liquid in a stirred reaction vessel heated in an oil bath. The apparatus operates on a closed loop, with a gas vent leading from the reaction vessel back into the dropping funnel, from the upper portion of which products are removed via a water condenser.

Two binary type ionic liquids were prepared from the addition of triflic acid to 1-butylpyridinium triflate, or to 3-(3-methylimidazolium-1-yl)propane-1-sulfonate (also known as MIPS) as shown in the following reaction scheme.

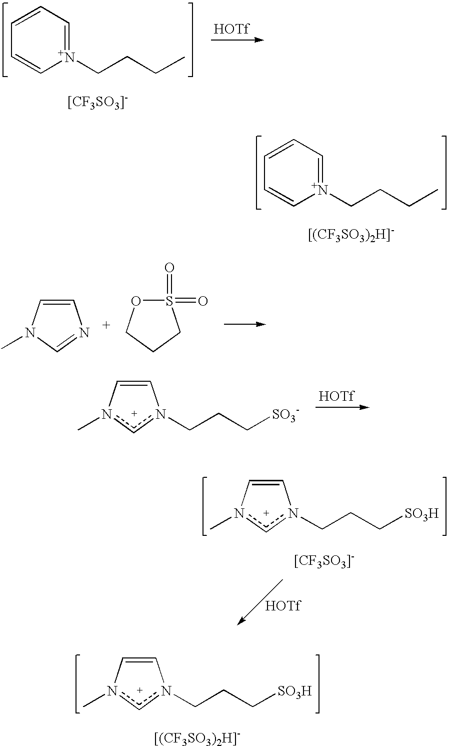

### EXAMPLE 7

### Methanol Dehydration

Methanol (32 g, 1.0 mol) was dropped onto the ionic liquid comprising [BuPy][OTf] (20 mmol, 5.71 g))/HOTf, ratio=2.0 (6.00 g), at 250 deg. C. The methanol vaporised on contact with the ionic liquid and was distilled back into the dropping funnel along with the water by-product. The product passed into a receiver flask cooled with a dry ice/acetone bath, via the top of the condenser and was collected, weighed and analysed by NMR. After 6 hours, dimethyl ether was obtained in a yield of 35%.

### EXAMPLE 8

### Methanol Dehydration

Methanol (32 g, 1.0 mol) was dropped onto the ionic liquid comprising 3-(3-methylimidazolium-1-yl)propane-1-sulfonate (10 mmol, 5.71 g))/HOTf, ratio=1.5 (2.25 g), at 250 deg. C., using the method of Example 7. After 5 hours, dimethyl ether was obtained in a yield of 42%.

In Examples 7 and 8, water tended to build up in the dropping funnel, and this reduced the reaction rate as the reaction proceeded. With a device to separate the water (by-product) from the methanol (reagent), the yields could be improved considerably.

### EXAMPLE 9

### Ethanol Dehydration

Absolute ethanol (46.1 g) was dropped onto the ionic liquid [MIPS]/[HOTf] (1:1.5) (10 mmol/15 mmol) at 240 to 260 deg. C. The product was collected in a Schlenk flask attached to the outlet of the condenser and cooled with liquid nitrogen. After 4 hours, 3.24 g ethene was collected in the Schlenk flask (along with 2.17 g diethyl ether and ethanol), corresponding to a yield of 12% of ethene.

Again, the water by-product inhibited this reaction. Higher temperatures and a water separation step would improve the yield.

### EXAMPLE 10

### Isopropanol Dehydration

Isopropanol (30.0 g, 0.50 mol) was dropped onto the ionic liquid [MIPS]/[HOTf] (1:1.5) (10 mmol/15 mmol) at 240 to 260 deg. C. The product was collected in a round bottom flask attached to the outlet of the condenser and cooled with dry ice and acetone. After 4 hours, 12.39 g propene, corresponding to a yield of 59%, was collected in the Schlenk flask (along with 2.44 g unreacted isopropanol and water). Very little diisopropyl ether (bp=68 deg. C.) was observed by NMR.

### EXAMPLE 11

### Pentanol Dehydration

The method of Example 10 was repeated using pentan-1-ol instead of isopropanol After 4 hours, 19.1 g of isomeric pentenes, corresponding to a yield of 55%, were collected. Very little diisopropyl ether (b p=68 deg. C.) was observed by NMR. The pentene isomers were present in the following amounts:

| | |
|---|---|
| pent-1-ene | 9 |
| cis-pent-2-ene | 26 |
| trans-pent-2-ene | 51 |
| 2-methylbut-1-ene | 3 |
| 3-methylbut-1-ene | 0 |
| 2-methylbut-2-ene | 9 |

### EXAMPLE 12

### Methanol Dehydration Using Heterogeneous Catalyst

The ionic liquid [MIPS]/HOTf (1:1.5) was supported on flash silica by mixing a methanol solution (50 ml) of the ionic liquid (8.0 g) with 20 g of silica. The methanol was evaporated and the supported ionic liquid heated at 90 deg. C. for 6 hours. The resulting product contained 40% ionic liquid.

The supported catalyst was heated to 200 deg. C. in a tube in a furnace, and methanol was passed over the catalyst at a rate of 20 ml/hr using a syringe pump. Product was collected in a sample tube. The apparatus (FIG. 2) was used and the product collected in a cooled sample tube at −78 deg. C. After 0.5 hrs., the products contained 23% dimethyl ether.

1-26. (canceled)

27. A process for producing an olefin and/or an ether which comprises heating an alcohol selected from methanol and/or ethanol in the presence of an acidic ionic compound which exists in a liquid state at a temperature of below 150° C.

28. A process as claimed in claim 27, in which the alcohol is methanol.

29. A process as claimed in claim 27, which is carried out under conditions such that the olefin and/or ether product and the co-produced water are formed in a vapour phase, and in which the co-produced water is condensed out from the olefin and/or ether product.

30. A process as claimed in claim 29, in which the alcohol is methanol.

31. A process as claimed in claim 29, which is carried out under conditions such that the olefin and/or ether product and the co-produced water are formed in a vapour phase separate from a liquid phase comprising said ionic compound, and in which the co-produced water is condensed out from the olefin and/or ether product.

32. A process as claimed in claim 27, in which said ionic compound exists in a liquid state at a temperature of below 100° C.

33. A process as claimed in claim 27, carried out at a temperature in the range 100 to 400° C.

34. A process as claimed in claim 27, in which the ionic liquid contains an ionic cation of the formula Cat⁺-Z-Acid wherein Cat⁺ is a cationic species; Z is a linking group joining Cat⁺ and Acid which may be a covalent bond or a group containing 1 to 30 carbon atoms and optionally one, two or three oxygen atoms; and Acid is an acidic moiety.

35. A process as claimed in claim 34, in which said ionic compound contains an acidic cation which is a quaternary ammonium or phosphonium cation of the general formula: 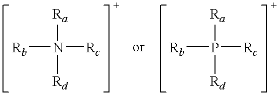
where each of Rₐ R_{b} R_{c} and R_{d} are independently selected from H, an alkyl group having from 1 to 30 carbon atoms, which may be optionally interrupted by 1, 2 or 3 carbon atoms, an aryl group, or a group —Z-Acid, at least one of Rₐ R_{b} R_{c} and R_{d} representing a group —Z-Acid; or in which said ionic compound comprises a group Cat⁺ which comprises or consists of a heterocyclic ring structure selected from imidazolium, pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium, diazabicyclo[5,4,0]undecenium, diazabicyclo[4,3,0]nonenium and pyrrolidinium.

36. A process as claimed in claim 35, in which Cat⁺ comprises or consists of a heterocyclic ring structure selected from imidazolium, pyridinium, pyrazolium, isothiazolinium, triazolium, tetrazolium, piperidinium, morpholinium, diazabicyclo[5,4,0]undecenium, diazabicyclo[4,3,0]nonenium and pyrrolidinium.

37. A process as claimed in claim 36, in which Cat⁺ comprises an or consists of an imidazolium heterocyclic ring structure.

38. A process as claimed in claim 34, in which said ionic compound comprises an acidic cation Cat⁺-Z-Acid which is selected from: 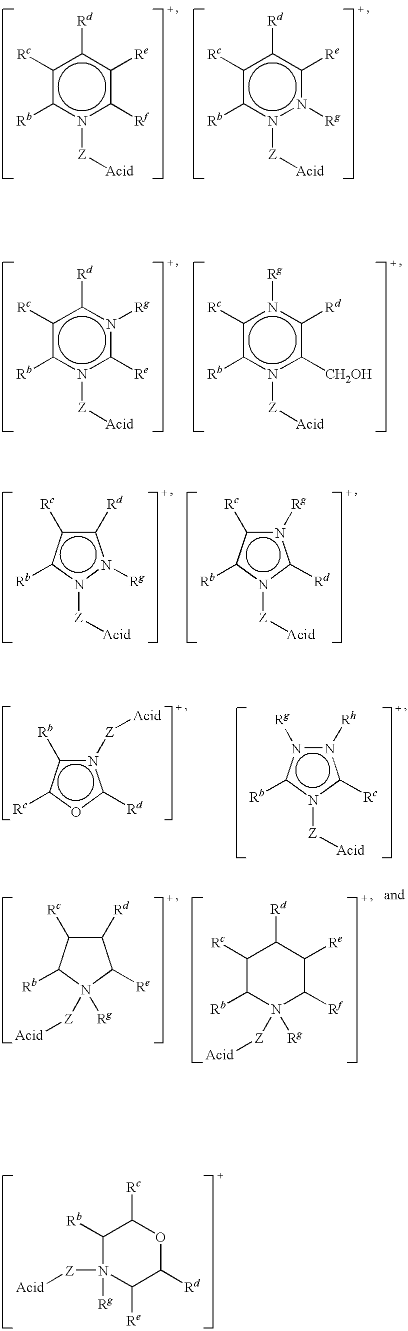
wherein Acid and Z are carried out at a temperature in the range of 100 to 400° C.; and R^{b}, R^{c}, R^{d}, R^{e}, R^{f}, R^{g} and R^{h} are each independently selected from hydrogen, a C₁ to C₄₀ alkyl group, a C₃ to C₈ cycloalkyl group, or a C₆ to C₁₀ aryl group, wherein said alkyl, cycloalkyl or aryl groups are unsubstituted or may be substituted by one to three groups selected from C₁ to C₆ alkoxy, C₆ to C₁₀ aryl, CN, OH, NO₂, C₇ to C₃₀ aralkyl, and C₇ to C₃₀ alkaryl, or any two of R^{b}, R^{c}, R^{d}, R^{e} and R^{f} attached to adjacent carbon atoms may form a methylene chain —(CH₂)_{q} wherein q is from 3 to 6.

39. A process as claimed in claim 34, in which Acid is selected from —SO₃H, —CO₂H, HSO₃-Ph-, HSO₃-Ph(R)—, —PO(OH)₂, —PO(OH), and —PO. R.(OH); wherein R is a C₁ to C₆ alkyl or haloalkyl group or an aryl group bearing one or more inert substituents.

40. A process as claimed in claim 27, in which said ionic compound contains an acidic anion selected from [HSO₄]⁻, [H₂PO₄]⁻, [HPO]²⁻, and [HX₂]⁻ wherein X=F, Cl, Br or I.

41. A process as claimed in claim 40, in which said ionic compound contains an acidic anion selected from [HCl₂]⁻, [HF₂]⁻, [HSO₄]⁻ and [H₂PO₄]⁻.

42. A process as claimed in claim 27, in which said ionic compound contains, as cation, choline, a C₆-₁₈alkyl-3-methylimidazolium cation, or a 4-(3-methylimidazolium)-butanesulfonate cation.

43. A process as claimed in claim 34, in which said ionic compound contains a neutral cation of the general formula NRₐR_{b}R_{c}R_{d}⁺ or PRₐR_{b}R_{c}R_{d}⁺ in which each of Rₐ R_{b} R_{c} and R_{d} is independently selected from H, an alkyl group having from 1 to 30 carbon atoms, which may be optionally interrupted by 1, 2 or 3 carbon atoms, or an aryl group; or a neutral cation comprising or consisting of a heterocyclic ring structure selected from imidazolium, pyridinium, pyrazolium, thiazolium, isothiazolinium, azathiozolium, oxothiazolium, oxazinium, oxazolium, oxaborolium, dithiazolium, triazolium, selenozolium, oxaphospholium, pyrollium, borolium, furanium, thiophenium, phospholium, pentazolium, indolium, indolinium, oxazolium, isooxazolium, isotriazolium, tetrazolium, benzofuranium, dibenzofuranium, benzothiophenium, dibenzothiophenium, thiadiazolium, pyrimidinium, pyrazinium, pyridazinium, piperazinium, piperidinium, morpholinium, pyranium, annolinium, phthalazinium, quinazolinium, quinazalinium, quinolinium, isoquinolinium, thazinium, oxazinium, azaannulenium-diazabicyclo[5,4,0]undecenium, diazabicyclo[4,3,0]nonenium, and pyrrolidinium.

44. A process as claimed in claim 43, in which said neutral cation comprises or consists of a heterocyclic ring structure selected from pyridinium, pyrazolium, thiazolium, pyrimidinium, piperazinium, piperidinium, morpholinium, quinolinium, isoquinolinium, diazabicyclo[5,4,0]undecenium, diazabicyclo [4,3,0]nonenium, and pyrrolidinium.

45. A process as claimed in claim 43, in which said neutral cation is selected from: 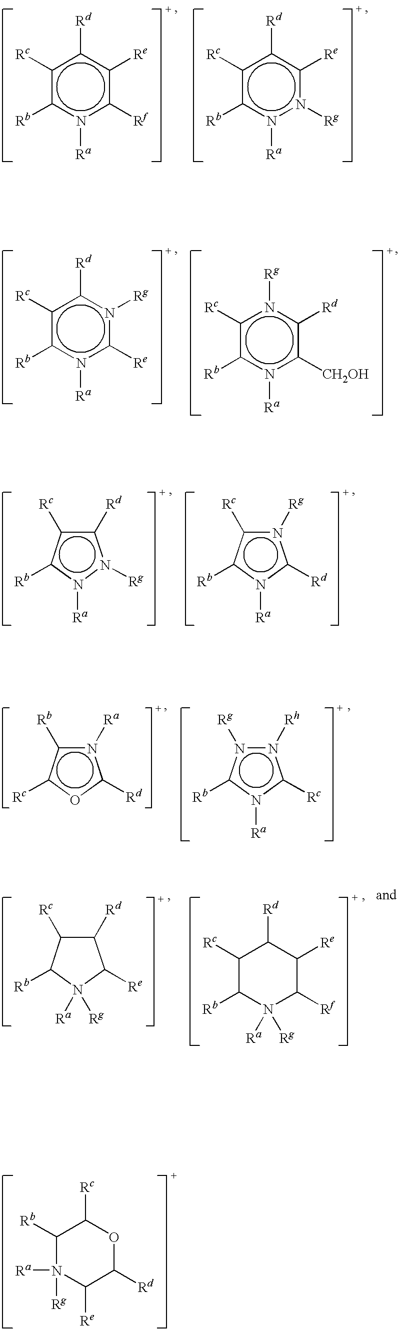
wherein R^{a}, R^{b}, R^{c} R^{d}, R^{e}, R^{f}, R^{g} and R^{h} are each independently selected from hydrogen, a C₁ to C₄₀ alkyl group, a C₃ to C₈ cycloalkyl group, or a C₆ to C₁₀ aryl group, wherein said alkyl, cycloalkyl or aryl groups are unsubstituted or may be substituted by one to three groups selected from C₁ to C₆ alkoxy, C₆ to C₁₀ aryl, CN, OH, NO₂, C₇ to C₃₀ aralkyl and C₇ to C₃₀ alkaryl, or any two of R^{b}, R^{c}, R^{d}, R^{e} and R^{f} attached to adjacent carbon atoms form a methylene chain —(CH₂)_{q}— wherein q is from 3 to 6.

46. A process as claimed in claim 34, wherein said ionic compound comprises a neutral anion selected from carboxylate, hydrogensulfate, sulfonate, phosphinate, triflamide, triflate, dicyanamide, oxide or halide.

47. A process as claimed in claim 46, in which said neutral anion is selected from [C(CN)₃]⁻, [NTf₂]⁻, [OTf]⁻, [R—SO₃]⁻, [R₂PO₂]⁻, [Cl]⁻, [Br]⁻ and [I]⁻, wherein R is C₁ to C₆ alkyl, C₆ to C₁₀ aryl, or C₇, to C₁₂ alkaryl.

48. A process as claimed in claim 37, in which said ionic compound contains an anion selected from dihydrogenphosphate, hydrogensulfate, trifluromethanesulfonate, bistrifluoromethanesulfonylamide, tosylate and metal anions [MClₘ] where M is gallium or indium.

49. A process as claimed in claim 27, in which said ionic compound is a binary acidic compound prepared by mixing an acidic compound with a salt of formula [Cation] [Anion].

50. A process as claimed in claim 49, in which said acidic compound is a strong mineral or organic acid or a metal halide or metal complex that exhibits Lewis acidity in which the metal is as transition metal, Group 13, 14, 15, 16 metal or semi metal, or a lanthanide or actinide.

51. A process as claimed in claim 50, in which said acidic compound is selected from sulfonic acids, fluorinated sulfonic acids, phosphoric acids, hydrogen sulfonamides, alkylsulfonic acids and haloacids, or is an aluminium, gallium or indium compound having Lewis acid properties.

52. A process as claimed in claim 49, in which the anion used to form said binary compound is selected from [C(CN)₃]⁻, [NTf₂]⁻, [OTf]⁻, [R—SO₃)⁻, [R₂PO₂]⁻, [Cl]⁻, [Br]⁻ and [I]⁻, wherein R is C₁ to C₆ alkyl, C₆ to C₁₀ aryl, or C₇ to C₁₂ alkaryl.

53. A process as claimed in claim 27, in which said ionic compound is choline dihydrogenphosphate, choline hydrogensulfate, hexylmethylimidazolium hydrogensulfate, [4-(3-methylimidazolium-1-yl)-butane-1-sulfonate] [(CF₃SO₂)₂N]), [4-(3-methylimidazolium-1-yl)-butane-1-sulfonate] [CF₃SO₃], [4-(3-methylimidazolium-1-yl)-butane-1-sulfonate] [CH₃C₆H₄SO₃], [4-(3-methylimidazolium-1-y])-butane-1-sulfonate] [H₂PO₄], N-butylpyridinium triflate, [3-(3-methylimidazolium-1-yl)propane-1-sulfonate)] [(CF₃SO₂)₂H], or [4-(3-methylimidazolium-1-yl)-butane-1-sulfonate] [(CF₃SO₂)₂N].
